(19) United States
(12) Patent Application Publication
(10) Pub. No.: US 2003/0229922 A1
(43) Pub. Date: Dec. 11, 2003

(54) Materials and methods for the modification of plant cell wall polysaccharides

(75) Inventor: Leonard N. Bloksberg, Remuera (NZ)

Correspondence Address: SPECKMAN LAW GROUP, 1501 WESTERN AVE, SUITE 100, SEATTLE, WA, 98101, US

(73) Assignees: GENESIS RESEARCH AND DEVELOPMENT CORPORATION LTD., Parnell (NZ); RUBICON FORESTS HOLDINGS LIMITED

(21) Appl. No.: 10/393,840

(22) Filed: Mar. 20, 2003

Related U.S. Application Data
(63) Continuation-in-part of application No. 09/636,800, filed on Aug. 10, 2000, now abandoned.
Application No. 09/636,800 is a continuation-in-part of application No. 09/170,862, filed on Oct. 13, 1998, now abandoned.
(60) Provisional application No. 60/148,426, filed on Aug. 11, 1999.

(51) Int. Cl. A01H 1/00; C12N 15/82; A01H 7/00; C07H 21/04; C12N 9/24; C12N 9/42; C12N 5/04

(52) U.S. Cl. 800/284; 800/319; 536/23.2; 435/69.1; 435/320.1; 435/200; 435/419; 435/209

(57) ABSTRACT

Novel isolated polynucleotides and polypeptides associated with the synthesis of plant cell wall polysaccharides are provided, together with genetic constructs comprising such sequences. Methods for using such constructs for the modulation of polysaccharide content in plants are also disclosed, together with transgenic plants comprising such constructs.

### REFERENCE TO RELATED APPLICATIONS

This application is a continuation-in-part of U.S. patent application Ser. No. 09/636,800 filed Aug. 10, 2000, which is a continuation-in-part of U.S. patent application Ser. No. 09/170,862 filed Oct. 13, 1998, and claims priority to U.S. Provisional Patent Application No. 60/148,426 filed Aug. 11, 1999.

### TECHNICAL FIELD OF THE INVENTION

This invention relates to the field of modification of cell wall polysaccharide content and composition in plants. More particularly, this invention relates to enzymes involved in the synthesis of plant cell wall polysaccharides and nucleotide sequences encoding such enzymes.

### BACKGROUND OF THE INVENTION

Plant cells are characterized by having a rigid cell wall. These cell walls are comprised primarily of polymers of simple sugar monomers linked in a variety of linear or branched polymers known as polysaccharide. The most abundant simple sugar monomer is glucose, and the most abundant polymer is cellulose. Cellulose is a linear, unbranched polymer, comprised of β-1,4 linked glucose monomers. Other polysaccharides found in plant cell walls include hemicellulose, which is a group of polysaccharides comprised of β-1,4 linked glucose monomers having side chains which may include sugars other than glucose. These side chains frequently include xylose, fucose, arabinose, and galactose. Pectins are another type of polysaccharide found in plant cell walls. Pectins are acidic polysaccharides, which are generally comprised primarily of galacturonic acid and rhamnose sugar monomers. Amylose is an additional common plant polysaccharide which is not usually found as a major component of cell walls. It acts primarily as a storage material for glucose, rather than as a structural polymer. However, because amylose is comprised primarily of α-1,4-linked glucose monomers, it is considered to be a related polymer from a biochemical and physiological perspective.

Plant polysaccharides have many uses. Certain plastics, such as cellulose acetate, and synthetic textiles, such as rayon, are made from cellulose. In addition, some biodegradable plastics and digestible medicine capsules, as well as medical fillers and fiber additives for food, can be made from plant polysaccharides.

In foodstuffs, polysaccharides have a profound impact on food quality. Cell walls contribute to crispness in carrots, while degradation of cell walls is required for softening of fruits, such as peaches and tomatoes. In maize, increased amylose is desirable for cattle feed, but not for human consumption, and increased cell wall strength reduces digestibility. In fiber crops, such as timber, cellulose is the primary polymer of interest. Wood density, a fundamental measure of structural timber quality, is essentially a measure of cellulose content. In the paper pulping industry, efficiency is measured in terms of yield of cellulose. Clearly, the ability to increase cellulose content in timber is an important economic goal.

The sugars which make up plant cell wall polysaccharides are produced in the photosynthetic organs of plants. The sugars so produced are commonly converted into sucrose, a disaccharide consisting of glucose and fructose. Sucrose is transported throughout the plant, to wherever sugar monomers are called for. Thus, the photosynthetic organs are often referred to as a source, while tissues requiring large amounts of sugar monomers are referred to as a sink. Actively growing regions of the plant are generally sink tissues, as new cell wall synthesis requires large amounts of sugar monomers.

When the transported sucrose arrives at the sink destination, it must be converted into whichever kind of sugar monomer is required. The sugar monomers which make up plant cell walls are primarily 5- or 6-carbon sugars. Different sugars are generally distinguished by stereospecific orientation of hydroxyl groups. Plants contain a variety of enzymes, such as isomerases or epimerases, which can rapidly change the orientation of these hydroxyls. In addition, there are a number of enzymes which can add or remove a single carbon from a sugar monomer. The result is a single pool of sugar monomers which the plant can freely inter-convert into whichever kind is needed for cell wall synthesis.

Plant polysaccharides are thus biochemically and physiologically inter-related. All polymers compete for the same pool of sugar monomers, and all sugar monomers can be freely interconverted to other types. Degradation of any one polymer will provide building material for any other. Attempts to engineer changes in one polymer may therefore have pleiotropic effects on other polymers.

The rate of cell wall synthesis is dependent on both the availability of sugar monomers to serve as building blocks for the polymers of the wall, and the enzymes which polymerise those building blocks into polymers. Enzymes which are directly responsible for the synthesis of the major cell wall polymers, such as cellulose, hemicellulose and pectin, may have a profound impact on the rate of cell wall synthesis. Source-sink relations may play an important role in limiting cell wall synthesis, if the availability of substrates becomes limiting. Polymer degrading enzymes may liberate sugar monomers from unnecessary polymers for use in building new, desired polymers. Enzymes which can isomerise sugars from one form into another can convert the sugars into whichever kind is needed. Each of the different types of cell wall polysaccharides effectively competes for the same pool of sugar monomers, and each represents a potential source of monomers for any of the other polymers.

The final committed steps in cellulose biosynthesis involve a relatively small number of enzymes. Cellulose synthase (CEL) is believed to function as part of a large, membrane-bound complex which also includes sucrose synthase (SUS: Amor et al., Proc. Natl. Acad. Sci USA 92:9353-9357, 1995) and annexin (ANX: Clark and Roux, Plant Phys. 109:1133-1139, 1995). This enzyme complex polymerises activated glucose into the cellulose polymer. The glucose is activated by UDP-glucose pyrophosphorylase (UGP), also known as UTP-glucose-1-phosphate uridylyltransferase. These enzymes are believed to be sufficient for the biosynthesis of cellulose from glucose. Other than these steps, the availability of glucose appears to be the most significant rate-limiting step in cellulose biosynthesis.

Glucose is primarily stored in most plants as amylose. Plants routinely store amylose and degrade it to free up the glucose monomers, as needed. By inhibiting the efficiency of glucose storage, or by increasing the liberation of glucose from amylose, the availability of glucose monomers for cellulose biosynthesis can be increased. The rate-limiting enzyme in the storage of glucose as amylose is ADP-glucose pyrophosphorylase (AGP), also known as ATP-glucose-1-phosphate adenylyltransferase (Iglesias et al., J. Biol. Chem. 268:1081-1086, 1993). Conversely, the enzyme most responsible for liberating glucose from amylose is amylase (AMA: Kawagoe and Delmer, Genetic Engineering 19:63-87, 1997).

These enzymes clearly will be important in the engineering of economically useful changes in cellulose biosynthesis. In addition, there are many other enzymes which may be useful in influencing plant cell wall polysaccharide biosynthesis. Other enzymes likely to be involved in cellulose biosynthesis include 1,4-β-cellobiohydrolase, β-glucosidase, calnexin, cellobiose epimerase, cellobiose phosphorylase, cellulase A, dextransucrase, invertase, phosphodiesterase, phosphoglucomutase, sucrose phosphate synthase, sucrose phosphorylase, UDP-glucose 4-epimerase and UDP-glucose dehydrogenase. Enzymes believed to be involved in hemicellulose biosynthesis include β-glucanase, arabinan synthase, GDP-fucose pyrophosphorylase, GDP-mannose pyrophosphorylase, 1,3 and 1,4-β-glucanases, 1,3 and 1,4-β-glucosidases, mannose-6-phosphate isomerase, nDP-hexose pyrophosphorylase, xyloglucan endotransglycosylase and xyloglucan synthase. Enzymes likely to be involved in pectin biosynthesis include α-galactosidase, β-glucuronidase, exopolygalacturonase, glucuronosyl-transferase, pectin methyl-esterase, polygalacturonase and UDP-hexose-1-phosphate uridylyltransferase. Enzymes believed to be involved in amylose biosynthesis include α-glucosidase, amylopectin 6-glucanohydrolase, amylopectin-branching glycosyltransferase, β-amylase, branching enzyme, inulosucrase, isoamylase, isomaltase, levansucrase, starch phosphorylase and starch synthase. Enzymes likely to be involved in the interconversion of 5-carbon sugars include 2-dehydro-3-deoxy-gluconokinase, aldehyde reductase, arabinose isomerase, D-arabinitol dehydrogenase, D-xylulose reductase, endo-1,4-β-xylanase, exo-1,4-β-xylanase, L-arabinose isomerase, L-ribulokinase, L-xylulokinase, phospho-ribulokinase, ribose 5-phosphate isomerase, ribulose-phosphate-3-epimerase, ribulose-phosphate-4-epimerase, transaldolase, transketolase, xylose isomerase and xylulokinase. Enzymes likely to be involved in interconversion of 6-carbon sugars include 6-phospho-fructo-1-kinase, 6-phospho-fructo-2-kinase, trehalose phosphate synthase, aldolase, aldose 1-epimerase, D-fructokinase, D-galactokinase, fructose 1,6-diphosphatase, gluconolactonase, glucose 1-phosphatase, glucose 6-phosphatase, glucose 6-phosphate dehydrogenase, glucose-phosphate isomerase, hexokinase, phosphoglucomutase, trehalase, trehalose phosphatase and UDP-galactose dehydrogenase.

While DNA sequences encoding some of the enzymes involved in the biosynthetic pathways of plant cell wall polysaccharides have been isolated for certain species of plants, genes encoding many of the enzymes in a wide range of plant species have not yet been identified. Thus, there remains a need in the art for materials useful in the modification of cell wall polysaccharide content and composition in plants.

### SUMMARY OF THE INVENTION

Briefly, the present invention provides polynucleotides isolated from eucalyptus and pine which encode enzymes involved in the synthesis of cell wall polysaccharides. Genetic constructs including such sequences and methods for the use of such constructs are also provided, together with transgenic plants having altered cell wall polysaccharide content and composition.

In one embodiment, the isolated polynucleotides comprise a nucleotide sequence selected from the group consisting of: (a) sequences recited in SEQ ID NOS: 1-29, 57-80, 105, 107, 109-113, 119-128, 139-143, 149-933 and 955; (b) complements of the sequences recited in SEQ ID NOS: 1-29, 57-80, 105, 107, 109-113, 119-128, 139-143, 149-933 and 955; (c) reverse complements of the sequences recited in SEQ ID NOS: 1-29, 57-80, 105, 107, 109-113, 119-128, 139-143, 149-933 and 955; (d) reverse sequences of the sequences recited in SEQ ID NOS: 1-29, 57-80, 105, 107, 109-113, 119-128, 139-143, 149-933 and 955; and (e) sequences having either 75%, 80%, 90%, 95% or 98% identity, as defined herein, to a sequence of (a)-(d).

In a further aspect, isolated polypeptides encoded by a polynucleotide of the present invention are provided. In specific embodiments, such polypeptides comprise an amino acid sequence selected from the group consisting of SEQ ID NOS: 30-56, 81-104, 106, 108, 114-118, 129-138, 144-148, 934-954 and 956, and variants thereof.

In another aspect, the invention provides genetic constructs comprising a polynucleotide of the present invention, either alone, in combination with one or more of the inventive polynucleotide sequences, or in combination with one or more known polynucleotides, together with transgenic cells comprising such constructs.

In a related aspect, the present invention provides genetic constructs comprising, in the 5′-3′ direction, a gene promoter sequence; an open reading frame coding for at least a functional portion of a polypeptide encoded by a polynucleotide of the present invention or a variant thereof; and a gene termination sequence. The open reading frame may be orientated in either a sense or antisense direction. Genetic constructs comprising a non-coding region of a gene coding for a polynucleotide encoded by the above polynucleotides or a nucleotide sequence complementary to a non-coding region, together with a gene promoter sequence and a gene termination sequence, are also provided. Preferably, the gene promoter and termination sequences are functional in a host plant. More preferably, the gene promoter and termination sequences are those of the original polypeptide genes but others generally used in the art, such as the Cauliflower Mosaic Virus (CMV) promoter, with or without enhancers such as the Kozak sequence or Omega enhancer, and Agrobacterium tumefaciens nopalin synthase terminator may be usefully employed in the present invention. Tissue-specific promoters may be employed in order to target expression to one or more desired tissues. In a preferred embodiment, the gene promoter sequence provides for transcription in xylem. The genetic construct may further include a marker for the identification of transformed cells.

In a further aspect, transgenic plant cells comprising the genetic constructs of the present invention are provided, together with plants comprising such transgenic cells, and fruits, seeds, and progeny of such plants.

In yet another aspect, methods for modulating the polysaccharide content and composition of an organism, such as a plant, are provided, such methods including stably incorporating into the genome of the plant a genetic construct of the present invention. In a preferred embodiment, the target plant is a woody plant, preferably selected from the group consisting of eucalyptus, pine, acacia, poplar, sweetgum, teak and mahogany species, more preferably from the group consisting of pine and eucalyptus species, and most preferably from the group consisting of Eucalyptus grandis and Pinus radiata. In a related aspect, a method for producing a plant having modified cellulose content is provided, the method comprising transforming a plant cell with a genetic construct of the present invention to provide a transgenic cell and cultivating the transgenic cell under conditions conducive to regeneration and mature plant growth.

In yet a further aspect, the present invention provides methods for modifying the activity of a polypeptide in a plant, comprising stably incorporating into the genome of the plant a genetic construct of the present invention. In a preferred embodiment, the target plant is a woody plant, preferably selected from the group consisting of eucalyptus, pine, acacia, poplar, sweetgum, teak and mahogany species, more preferably from the group consisting of pine and eucalyptus species, and most preferably from the group consisting of Eucalyptus grandis and Pinus radiata.

The above-mentioned and additional features of the present invention and the manner of obtaining them will become apparent, and the invention will be best understood by reference to the following more detailed description. All references disclosed herein are hereby incorporated by reference in their entirety as if each was incorporated individually.

### BRIEF DESCRIPTION OF THE FIGURES

FIG. 1 illustrates the level of native CEL enzyme activity in positive control mung bean (V. radiata) plants.

FIG. 2 illustrates the level of CEL enzyme activity in mammalian 293T cells transfected with E. grandis CEL as compared to that in non-transfected 293T cells.

FIG. 3 shows the amino acid sequence of SEQ ID NO: 956, a cellulose synthase. The cDNA sequence encoding SEQ ID NO: 956 is provided in SEQ ID NO: 955. Eight transmembrane domains are underlined and the four conserved domains (U-1 to U-4) are boxed. A LIM-like Zn-binding domain/RING finger domain is boxed with conserved Cys residues in bold. Three conserved Asp residues are in bold and double-underlined and the conserved QXXRW motif in the U4 domain is in bold (Taylor et al., Plant Cell 12:2529-2539, 2000; Pear et al., Proc. Natl. Acad. Sci. USA 93:12637-12642, 1996; Richmond and Somerville, Plant Physiol. 124:495-498, 2000).

FIG. 4 shows the amino acid sequence of SEQ ID NO: 93, a cellulose synthase. The cDNA sequence encoding SEQ ID NO: 93 is provided in SEQ ID NO: 69. Eight transmembrane domains are underlined and the four conserved domains (U-1 to U-4) are boxed. A LIM-like Zn-binding domain/RING finger domain is boxed with conserved Cys residues in bold. Three conserved Asp residues are in bold and double-underlined and the conserved QXXRW motif in the U4 domain is in bold (Taylor et al., Plant Cell 12:2529-2539, 2000; Pear et al., Proc. Natl. Acad. Sci. USA 93:12637-12642, 1996; Richmond and Somerville, Plant Physiol. 124:495-498, 2000).

### DETAILED DESCRIPTION

As outlined above, cellulose is formed by polymerization of glucose into a linear, unbranched, polymer comprised of β-1,4 linked glucose monomers (Kawagoe and Delmer, Genetic Engineering, 19:63-87, 1997). Cellulose is the most important plant cell wall polysaccharide from both a structural, as well as industrial, perspective. Other polysaccharides are essential for healthy cell walls, as well as for many alternative industrial uses.

Glucose monomers are most commonly stored in the plant in the form of amylose by the action of several enzymes, with the rate limiting step for storage being catalysed by AGP (Iglesias et al., J. Biol. Chem. 268:1081-1086). Glucose monomers are freed from this storage polymer by the action of the enzyme AMA. The free monomers are activated by the action of the enzyme UGP, and polymerised into cellulose macro-crystalline structures by the action of the cellulose synthase enzyme complex. Pure CEL enzyme has been shown to form β-1,4 glucose linkages in vitro, but has not been shown to be sufficient for polymerization of the large polymers which are fundamental to the structure of plant cell walls. The holoenzyme complex appears to be necessary for this latter function. The holoenzyme is believed to be comprised of the CEL enzyme in combination with the SUS enzyme and ANX, the whole complex being integrated into the plasma membrane and forming a “rosette” structure as seen in electron micrographs of plant cell membranes (Arioli et al., Science 279:717-720, 1998).

Because cellulose synthesis can represent such a large sink for sugar monomers in the cell, changes in the rate of cellulose synthesis can have a profound influence on the synthesis of other plant polysaccharides. Conversely, changes in the rates of synthesis of other plant polysaccharides can have a profound influence on the pool of sugars available for synthesis of cellulose. Hence, changes in the synthesis of any single polymer may affect both the content and composition of plant cell wall polysaccharides, and polysaccharides in general.

Quantitative and qualitative modifications in plant polysaccharide content are known to be induced by external factors such as light stimulation, low calcium levels, and mechanical stress. Synthesis of cell wall polysaccharides can also be induced by infection with pathogens.

Using the methods and materials of the present invention, the polysaccharide content of a plant may be increased or reduced, by incorporating additional copies of genes encoding enzymes involved in the synthesis of cell wall polysaccharides into the genome of the target plant. Similarly, an increase or decrease in polysaccharide content may be obtained by transforming the target plant with antisense copies of such genes. In addition, the number of copies of genes encoding for different enzymes in the biosynthetic pathway of cell wall polysaccharides can be manipulated to modify the relative amount of each monosaccharide synthesized, thereby leading to the formation of cell walls having altered composition. The alteration of polysaccharide composition would be advantageous, for example, in tree processing for paper.

The polynucleotides of the present invention were isolated from forestry plant sources, namely from Eucalyptus grandis and Pinus radiata, but they may alternatively be synthesized using conventional synthesis techniques. Specifically, isolated polynucleotides of the present invention include polynucleotides comprising a sequence selected from the group consisting of sequences identified as SEQ ID NOS: 1-29, 57-80, 105, 107, 109-113, 119-128, 139-143, 149-933 and 955; complements of the sequences identified as SEQ ID NOS: 1-29, 57-80, 105, 107, 109-113, 119-128, 139-143, 149-933 and 955; reverse complements of the sequences identified as SEQ ID NOS: 1-29, 57-80, 105, 107, 109-113, 119-128, 139-143, 149-933 and 955; at least a specified number of contiguous residues (x-mers) of any of the above-mentioned polynucleotides; extended sequences corresponding to any of the above polynucleotides; antisense sequences corresponding to any of the above polynucleotides; and variants of any of the above polynucleotides, as that term is described in this specification.

In another embodiment, the present invention provides isolated polypeptides encoded by the DNA sequences of SEQ ID NOS: 1-29, 57-80, 105, 107, 109-113, 119-128, 139-143, 149-933 and 955. The amino acid sequences encoded by SEQ ID NOS: 1-22, 24-28, 57-80, 105, 107, 109-113, 119-143, 149-933 and 955 are provided in SEQ ID NOS: 30-56, 81-104, 106, 108, 114-118, 129-138, 144-148, 934-954 and 956, respectively. The present invention also encompasses polynucleotides that differ from the disclosed sequences but which, due to the degeneracy of the genetic code, encode a polypeptide which is the same as that encoded by a polynucleotide of the present invention.

The polynucleotides and polypeptides of the present invention were putatively identified by DNA and polypeptide similarity searches. In the attached Sequence Listing SEQ ID NOS: 1-29, 57-80, 105, 107, 109-113, 119-128, 139-143, 149-933 and 955 are polynucleotide sequences, and SEQ ID NOS: 30-56, 81-104, 106, 108, 114-118, 129-138, 144-148, 934-954 and 956 are polypeptide sequences. The polynucleotides and polypeptides of the present invention have demonstrated similarity to enzymes that are known to be involved in the synthesis of cell wall polysaccharides. The putative identity of each of the inventive polynucleotides and polypeptides is shown below in Table 1.

TABLE 1

| DNA | PROTEIN | |
|---|---|---|
| SEQ ID NO: | SEQ ID NO: | IDENTITY |
| 1 | 30 | AGP |
| 2 | 31 | AGP |
| 3 | 32 | AGP |
| 4 | 33 | AMA |
| 5 | 34 | AMA |
| 6 | 35 | AMA |
| 7 | 36 | CEL |
| 8 | 37 | CEL |
| 9 | 38 | CEL |
| 10 | 39 | CEL |
| 11 | 40 | CEL |
| 12 | 41 | CEL |
| 13 | 42 | CEL |
| 14 | 43 | CEL |
| 15 | 44 | SUS |
| 16 | 45 | SUS |
| 17 | 46 | SUS |
| 18 | 47 | SUS |
| 19 | 48 | SUS |
| 20 | 49 | UGP |
| 21 | 50 | UGP |
| 22 | 51 | UGP |
| 23 | — | UGP |
| 24 | 52 | ANX |
| 25 | 53 | ANX |
| 26 | 54 | ANX |
| 27 | 55 | ANX |
| 28 | 56 | ANX |
| 29 | — | ANX |
| 57 | 81 | AMA |
| 58 | 82 | AMA |
| 59 | 83 | AGP |
| 60 | 84 | AGP |
| 61 | 85 | AGP |
| 62 | 86 | AGP |
| 63 | 87 | AGP |
| 64 | 88 | AGP |
| 65 | 89 | AGP |
| 66 | 90 | CEL |
| 67 | 91 | CEL |
| 68 | 92 | CEL |
| 69 | 93 | CEL |
| 70 | 94 | CEL |
| 71 | 95 | SUS |
| 72 | 96 | SUS |
| 73 | 97 | SUS |
| 74 | 98 | SUS |
| 75 | 99 | SUS |
| 76 | 100 | SUS |
| 77 | 101 | SUS |
| 78 | 102 | SUS |
| 79 | 103 | UGP |
| 80 | 104 | UGP |
| 105 | 106 | SUS |
| 107 | 108 | CEL |
| 109 | 114 | ANX |
| 110 | 115 | ANX |
| 111 | 116 | ANX |
| 112 | 117 | ANX |
| 113 | 118 | ANX |
| 119 | 129 | CEL |
| 120 | 130 | CEL |
| 121 | 131 | CEL |
| 122 | 132 | CEL |
| 123 | 133 | CEL |
| 124 | 134 | CEL |
| 125 | 135 | CEL |
| 126 | 136 | CEL |
| 127 | 137 | CEL |
| 128 | 138 | CEL |
| 139 | 144 | SUS |
| 140 | 145 | α-amylase |
| 141, 955 | 146, 956 | CEL |
| 142 | 147 | AGP (3′ end of SEQ ID NO: 62) |
| 143 | 148 | SUS (3′ of SEQ ID NO: 74) |
| 149-185 | — | 1,3-β-D-Glucanase |
| 186 | — | 1,4-β-Cellobiohydrolase |
| 187-196 | — | α,α-trehalose phosphate synthase |
| 197-204 | — | α-glucosidase |
| 205-250 | — | aldolase |
| 251 | — | Amylopectin 6-glucanohydrolase |
| 252-262 | — | β-amylase |
| 263 | — | β-glucosidase |
| 264-272 | — | Branching enzyme |
| 273-318 | — | D-fructokinase |
| 319-354 | — | D-xylulose reductase |
| 355-365 | — | Endo-1,3-1,4-β-glucanase |
| 366-371 | — | Glucan exo-1,3-β-glucosidase |
| 372-377 | — | Glucose 6-phosphate dehydrogenase |
| 378-381 | — | Glucose phosphate isomerase |
| 382-389 | — | Isoamylase |
| 390-393 | — | L-ribulokinase |
| 394-398 | — | Mannitol-1-phosphate 5-dehydrogenase |
| 399-478 | — | Pectin methyl-esterase |
| 479-506 | — | Phosphoglucomutase |
| 507-508 | — | Phospho-ribulokinase |
| 509-521 | — | Ribulose-phosphate-3-epimerase |
| 522-530 | — | Starch phosphorylase |
| 531-551 | — | Sucrose phosphate synthase |
| 552-555 | — | SUS |
| 556-586 | — | Transketolase |
| 587-591 | — | Trehalase |
| 592-620 | — | UDP-glucose 4-epimerase |
| 621-902 | — | Xyloglucan endotransglycosylase |
| 903-908 | — | Xylose isomerase |
| 903-908 | — | Xylose isomerase |
| 909 | — | Sucrose synthase |
| 910 | — | D-fructokinase |
| 911 | — | Xylose isomerase |
| 912 | — | Cellulose synthase |
| 913-921 | 934-942 | Cellulose synthase |
| 922-927 | 943-948 | Sucrose synthase |
| 928-929 | 949-950 | Alpha amylase |
| 930-933 | 951-954 | Annexin |

The term “polynucleotide(s),” as used herein, means a single or double-stranded polymer of deoxyribonucleotide or ribonucleotide bases and includes DNA and corresponding RNA molecules, including HnRNA and mRNA molecules, both sense and anti-sense strands, and comprehends cDNA, genomic DNA and recombinant DNA, as well as wholly or partially synthesized polynucleotides. An HnRNA molecule contains introns and corresponds to a DNA molecule in a generally one-to-one manner. An mRNA molecule corresponds to an HnRNA and DNA molecule from which the introns have been excised. A polynucleotide may consist of an entire gene, or any portion thereof. Operable anti-sense polynucleotides may comprise a fragment of the corresponding polynucleotide, and the definition of “polynucleotide” therefore includes all such operable anti-sense fragments.

The term “polypeptide”, as used herein, encompasses amino acid chains of any length including full length proteins, wherein amino acid residues are linked by covalent peptide bonds. Polypeptides of the present invention may be purified natural products, or may be produced partially or wholly using recombinant techniques.

The definition of the terms “complement”, “reverse complement” and “reverse sequence”, as used herein, is best illustrated by the following example. For the sequence 5′ AGGACC 3′, the complement, reverse complement and reverse sequence are as follows:

| | |
|---|---|
| complement | 3′ TCCTGG 5′ |
| reverse complement | 3′ GGTCCT 5′ |
| reverse sequence | 5′ CCAGGA 3′. |

Preferably, sequences that are complements of a specifically recited polynucleotide sequence are complementary over the entire length of the specific polynucleotide sequence.

As used herein, the term “variant” comprehends nucleotide or amino acid sequences different from the specifically identified sequences, wherein one or more nucleotides or amino acid residues is deleted, substituted, or added. Variants may be naturally occurring allelic variants or non-naturally occurring variants. Variant sequences (polynucleotide or polypeptide) preferably exhibit at least 75%, more preferably at least 80%, more preferably yet at least 90%, more preferably at least 95% and most preferably, at least 98% identity to a sequence of the present invention. The percentage identity is determined by aligning the two sequences to be compared as described below, determining the number of identical residues in the aligned portion, dividing that number by the total number of residues in the inventive (queried) sequence, and multiplying the result by 100.

Polynucleotides and polypeptides having a specified percentage identity to a polynucleotide or polypeptide identified in herein thus share a high degree of similarity in their primary structure. In addition to a specified percentage identity to a polynucleotide of the present invention, variant polynucleotides and polypeptides preferably have additional structural and/or functional features in common with a polynucleotide of the present invention. Polynucleotides having a specified degree of identity to, or capable of hybridizing to, a polynucleotide of the present invention preferably additionally have at least one of the following features: (1) they contain an open reading frame, or partial open reading frame, encoding a polypeptide, or a functional portion of a polypeptide, having substantially the same functional properties as the polypeptide, or functional portion thereof, encoded by a polynucleotide in a recited SEQ ID NO:; or (2) they contain identifiable domains in common.

Polynucleotide or polypeptide sequences may be aligned, and percentages of identical nucleotides or amino acids in a specified region may be determined against another polynucleotide or polypeptide, using computer algorithms that are publicly available. The BLASTN and FASTA algorithms, set to the default parameters described in the documentation and distributed with the algorithm, may be used for aligning and identifying the similarity of polynucleotide sequences. The alignment and similarity of polypeptide sequences may be examined using the BLASTP algorithm. BLASTX and FASTX algorithms compare nucleotide query sequences translated in all reading frames against polypeptide sequences. The FASTA and FASTX algorithms are described in Pearson and Lipman, Proc. Natl. Acad. Sci. USA 85:2444-2448, 1988; and in Pearson, Methods in Enzymol. 183:63-98, 1990. The FASTA software package is available from the University of Virginia by contacting the Assistant Provost for Research, University of Virginia, PO Box 9025, Charlottesville, Va. 22906-9025. The BLASTN software is available from the National Center for Biotechnology Information (NCBI), National Library of Medicine, Building 38A, Room 8N805, Bethesda, Md. 20894. The BLASTN algorithm Version 2.0.11 [January 20, 2000] set to the default parameters described in the documentation and distributed with the algorithm, is preferred for use in the determination of polynucleotide variants according to the present invention. The use of the BLAST family of algorithms, including BLASTN, BLASTP and BLASTX, is described in the publication of Altschul et al., “Gapped BLAST and PSI-BLAST: a new generation of protein database search programs,” Nucleic Acids Res. 25:3389-3402, 1997.

The following running parameters are preferred for determination of alignments and similarities using BLASTN that contribute to the E values and percentage identity for polynucleotides: Unix running command with the following default parameters: blastall -p blastn -d embldb -e 10 -G O-E O-r 1 -v 30 -b 30 -i queryseq -o results; and parameters are: -p Program Name [String]; -d Database [String]; -e Expectation value (E) [Real]; -G Cost to open a gap (zero invokes default behavior) [Integer]; -E Cost to extend a gap (zero invokes default behavior) [Integer]; -r Reward for a nucleotide match (BLASTN only) [Integer]; -v Number of one-line descriptions (V) [Integer]; -b Number of alignments to show (B) [Integer]; -i Query File [File In]; -o BLAST report Output File [File Out] Optional.

The following running parameters are preferred for determination of alignments and similarities using BLASTP that contribute to the E values and percentage identity of polypeptide sequences: blastall -p blastp d swissprottrembledb -e 10 -G O-E O-v 30 -b 30 -i queryseq -o results; the parameters are: -p Program Name [String]; -d Database [String]; -e Expectation value (E) [Real]; -G Cost to open a gap (zero invokes default behavior) [Integer]; -E Cost to extend a gap (zero invokes default behavior) [Integer]; -v Number of one-line descriptions (v) [Integer]; -b Number of alignments to show (b) [Integer]; -I Query File [File In]; -o BLAST report Output File [File Out] Optional.

The “hits” to one or more database sequences by a queried sequence produced by BLASTN, BLASTP, FASTA, or a similar algorithm, align and identify similar portions of sequences. The hits are arranged in order of the degree of similarity and the length of sequence overlap. Hits to a database sequence generally represent an overlap over only a fraction of the sequence length of the queried sequence.

As noted above, the percentage identity of a polynucleotide or polypeptide sequence is determined by aligning polynucleotide and polypeptide sequences using appropriate algorithms, such as BLASTN or BLASTP, respectively, set to default parameters; identifying the number of identical nucleic or amino acids over the aligned portions; dividing the number of identical nucleic or amino acids by the total number of nucleic or amino acids of the polynucleotide or polypeptide of the present invention; and then multiplying by 100 to determine the percentage identity. By way of example, a queried polynucleotide having 220 nucleic acids has a hit to a polynucleotide sequence in the EMBL database having 520 nucleic acids over a stretch of 23 nucleotides in the alignment produced by the BLASTN algorithm using the default parameters. The 23-nucleotide hit includes 21 identical nucleotides, one gap and one different nucleotide. The percentage identity of the 1 queried polynucleotide to the hit in the EMBL database is thus 21/220 times 100, or 9.5%. The percentage identity of polypeptide sequences may be determined in a similar fashion.

The BLASTN and BLASTX algorithms also produce “Expect” values for polynucleotide and polypeptide alignments. The Expect value (E) indicates the number of hits one can “expect” to see over a certain number of contiguous sequences by chance when searching a database of a certain size. The Expect value is used as a significance threshold for determining whether the hit to a database indicates true similarity. For example, an E value of 0.1 assigned to a polynucleotide hit is interpreted as meaning that in a database of the size of the EMBL database, one might expect to see 0.1 matches over the aligned portion of the sequence with a similar score simply by chance. By this criterion, the aligned and matched portions of the sequences then have a probability of 90% of being related. For sequences having an E value of 0.01 or less over aligned and matched portions, the probability of finding a match by chance in the EMBL database is 1% or less using the BLASTN algorithm. E values for polypeptide sequences may be determined in a similar fashion using various polypeptide databases, such as the SwissProt-TrEMBLE database.

According to one embodiment, “variant” polynucleotides and polypeptides, with reference to each of the polynucleotides and polypeptides of the present invention, preferably comprise sequences having the same number or fewer nucleotides or amino acids than each of the polynucleotides or polypeptides of the present invention and producing an E value of 0.01 or less when compared to the polynucleotide or polypeptide of the present invention. That is, a variant polynucleotide or polypeptide is any sequence that has at least a 99% probability of being related to the polynucleotide or polypeptide of the present invention, measured as having an E value of 0.01 or less using the BLASTN or BLASTX algorithms set at the default parameters. According to a preferred embodiment, a variant polynucleotide is a sequence having the same number or fewer nucleic acids than a polynucleotide of the present invention that has at least a 99% probability of being related to the polynucleotide of the present invention, measured as having an E value of 0.01 or less using the BLASTN algorithm set at the default parameters. Similarly, according to a preferred embodiment, a variant polypeptide is a sequence having the same number or fewer amino acids than a polypeptide of the present invention that has at least a 99% probability of being related as the polypeptide of the present invention, measured as having an E value of 0.01 or less using the BLASTP algorithm set at the default parameters.

In an alternative embodiment, variant polynucleotides are sequences that hybridize to a polynucleotide of the present invention under stringent conditions. Stringent hybridization conditions for determining complementarity include salt conditions of less than about 1 M, more usually less than about 500 mM, and preferably less than about 200 mM. Hybridization temperatures can be as low as 5° C., but are generally greater than about 22° C., more preferably greater than about 30° C., and most preferably greater than about 37° C. Longer DNA fragments may require higher hybridization temperatures for specific hybridization. Since the stringency of hybridization may be affected by other factors such as probe composition, presence of organic solvents, and extent of base mismatching, the combination of parameters is more important than the absolute measure of any one alone. An example of “stringent conditions” is prewashing in a solution of 6×SSC, 0.2% SDS; hybridizing at 65° C., 6×SSC, 0.2% SDS overnight; followed by two washes of 30 minutes each in 1×SSC, 0.1% SDS at 65° C. and two washes of 30 minutes each in 0.2×SSC, 0.1% SDS at 65° C.

The present invention also encompasses polynucleotides that differ from the disclosed sequences but that, as a consequence of the discrepancy of the genetic code, encode a polypeptide having similar enzymatic activity as a polypeptide encoded by a polynucleotide of the present invention. Thus, polynucleotides comprising sequences that differ from the polynucleotide sequences recited in SEQ ID NOS: 1-29, 57-80, 105, 107, 109-113, 119-128, 139-143, 149-933 and 955, or complements, reverse sequences, or reverse complements of those sequences, as a result of conservative substitutions are contemplated by and encompassed within the present invention. Additionally, polynucleotides comprising sequences that differ from the polynucleotide sequences recited in SEQ ID NOS: 1-29, 57-80, 105, 107, 109-113, 119-128, 139-143, 149-933 and 955, or complements, reverse complements, or reverse sequences as a result of deletions and/or insertions totaling less than 10% of the total sequence length are also contemplated by and encompassed within the present invention. Similarly, polypeptides comprising sequences that differ from the polypeptide sequences recited in SEQ ID NOS: 30-56, 81-104, 106, 108, 114-118, 129-138, 144-148, 934-954 and 956 as a result of amino acid substitutions, insertions, and/or deletions totaling less than 10% of the total sequence length are contemplated by an encompassed within the present invention, provided the variant polypeptide has activity in a cell wall polysaccharide synthesis pathway.

Variants of the polypeptide sequences recited in SEQ ID NOS: 30-56, 81-104, 106, 108, 114-118, 129-138, 144-148, 934-954 and 956 wherein the variant has an activity level that is different to that of the recited polypeptide are also encompassed by the present invention. In specific embodiments, variants of the inventive sucrose synthase (SUS) polypeptides are provided wherein the N-terminal serine phosphorylation site has been replaced by an acidic amino acid (such as Asp or Glu) by, for example, site directed mutagenesis. Nakai et al. have demonstrated that SUS polypeptides mutated in this manner possess increased activity compared to wild-type SUS (Nakai et al., Plant Cell Physiol. 39:1337-1341, 1998). Polynucleotides encoding such variants of the inventive SUS polypeptides may therefore be employed in transgenic plants to increase cellulose production.

The polynucleotides of the present invention may be isolated from various libraries, or may be synthesized using techniques that are well known in the art. The polynucleotides may be synthesized, for example, using automated oligonucleotide synthesizers (e.g., Beckman Oligo 1000M DNA Synthesizer) to obtain polynucleotide segments of up to 50 or more nucleic acids. A plurality of such polynucleotide segments may then be ligated using standard DNA manipulation techniques that are well known in the art of molecular biology. One conventional and exemplary polynucleotide synthesis technique involves synthesis of a single stranded polynucleotide segment having, for example, 80 nucleic acids, and hybridizing that segment to a synthesized complementary 85 nucleic acid segment to produce a 5 nucleotide overhang. The next segment may then be synthesized in a similar fashion, with a 5 nucleotide overhang on the opposite strand. The “sticky” ends ensure proper ligation when the two portions are hybridized. In this way, a complete polynucleotide of the present invention may be synthesized entirely in vitro.

Certain of the polynucleotides disclosed herein are referred to as “partial” sequences, in that they do not represent the full coding portion of a gene encoding a naturally occurring polypeptide. The partial polynucleotide sequences disclosed herein may be employed to obtain the corresponding full length genes for various species and organisms by, for example, screening DNA expression libraries using hybridization probes based on the polynucleotides of the present invention, or using PCR amplification with primers based upon the polynucleotides of the present invention. In this way one can, using methods well known in the art, extend a polynucleotide of the present invention upstream and downstream of the corresponding mRNA, as well as identify the corresponding genomic DNA, including the promoter and enhancer regions, of the complete gene. The present invention thus comprehends isolated polynucleotides comprising a sequence identified in SEQ ID NOS: 1-29, 57-80, 105, 107, 109-113, 119-128, 139-143, 149-933 and 955, or a variant of one of the specified sequences that encode a functional polypeptide, including full length genes. Such extended polynucleotides may have a length of from about 50 to about 4,000 nucleic acids or base pairs, and preferably have a length of less than about 4,000 nucleic acids or base pairs, more preferably yet a length of less than about 3,000 nucleic acids or base pairs, more preferably yet a length of less than about 2,000 nucleic acids or base pairs. Under some circumstances, extended polynucleotides of the present invention may have a length of less than about 1,800 nucleic acids or base pairs, preferably less than about 1,600 nucleic acids or base pairs, more preferably less than about 1,400 nucleic acids or base pairs, more preferably yet less than about 1,200 nucleic acids or base pairs, and most preferably less than about 1,000 nucleic acids or base pairs.

The polynucleotides identified herein may contain open reading frames (“ORFs”) or partial open reading frames encoding polypeptides and functional portions of polypeptides. Partial open reading frames are encoded by SEQ ID NOS: 1-14, 16-27, 29, 57, 60-68, 70-78, 105, 107, 109-113, 119-128, 140, 142, 143, 913-933, while SEQ ID NOS: 15, 28, 58, 59, 69, 79, 80, 139, 141, 152 and 955 represent full-length sequences. Additionally, open reading frames encoding polypeptides may be identified in extended or full length sequences corresponding to the sequences disclosed herein. Open reading frames may be identified using techniques that are well known in the art. These techniques include, for example, analysis for the location of known start and stop codons, most likely reading frame identification based on codon frequencies, etc. These techniques include, for example, analysis for the location of known start and stop codons, most likely reading frame identification based on codon frequencies, etc. Suitable tools and software for ORF analysis are well known in the art and include, for example, GeneWise, available from The Sanger Center, Wellcome Trust Genome Campus, Hinxton, Cambridge, CB10 1 SA, United Kingdom; Diogenes, available from Computational Biology Centers, University of Minnesota, Academic Health Center, UMHG Box 43 Minneapolis Minn. 55455; and GRAIL, available from the Informatics Group, Oak Ridge National Laboratories, Oak Ridge, Tennessee Tenn. Once a partial open reading frame is identified, the polynucleotide may be extended in the area of the partial open reading frame using techniques that are well known in the art until the polynucleotide for the full open reading frame is identified.

Once open reading frames are identified in the polynucleotides of the present invention, the open reading frames may be isolated and/or synthesized. Expressible genetic constructs comprising the open reading frames and suitable promoters, initiators, terminators, etc., which are well known in the art, may then be constructed. Such genetic constructs may be introduced into a host cell to express the polypeptide encoded by the open reading frame. Suitable host cells may include various prokaryotic and eukaryotic cells, including plant cells, mammalian cells, bacterial cells, algae and the like.

Polynucleotides of the present invention also comprehend polynucleotides comprising at least a specified number of contiguous residues (x-mers) of any of the polynucleotides identified as SEQ ID NOS: 1-29, 57-80, 105, 107, 109-113, 119-128, 139-143, 149-933 and 955, complements, reverse sequences, and reverse complements of such sequences, and their variants. Similarly, polypeptides of the present invention comprehend polypeptides comprising at least a specified number of contiguous residues (x-mers) of any of the polypeptides identified as SEQ ID NOS: 30-56, 81-104, 106, 108, 114-118, 129-138, 144-148 and 934-954 and their variants. As used herein, the term “x-mer,” with reference to a specific value of “x,” refers to a sequence comprising at least a specified number (“x”) of contiguous residues of any of the polynucleotides identified as SEQ ID NO: 1-29, 57-80, 105, 107, 109-113, 119-128, 139-143, 149-933 and 955, or the polypeptides identified as SEQ ID NOS: 30-56, 81-104, 106, 108, 114-118, 129-138, 144-148, 934-954 and 956. According to preferred embodiments, the value of x is preferably at least 20; more preferably, at least 40; more preferably yet, at least 60; and most preferably, at least 80. Thus, polynucleotides and polypeptides of the present invention comprise a 20-mer, a 40-mer, a 60-mer, an 80-mer, a 100-mer, a 120-mer, a 150-mer, a 180-mer, a 220-mer, a 250-mer, or a 300-mer, 400-mer, 500-mer or 600-mer of a polynucleotide or polypeptide identified as SEQ ID NOS: 1-956, and variants thereof.

Polynucleotide probes and primers complementary to and/or corresponding to SEQ ID NOS: 1-29, 57-80, 105, 107, 109-113, 119-128, 139-143, 149-933 and 955, and variants of those sequences, are also comprehended by the present invention. Such oligonucleotide probes and primers are substantially complementary to the polynucleotide of interest over a certain portion of the polynucleotide. As used herein, the term “oligonucleotide” refers to a relatively short segment of a polynucleotide sequence, generally comprising between 6 and 60 nucleotides, and comprehends both probes for use in hybridization assays and primers for use in the amplification of DNA by polymerase chain reaction.

An oligonucleotide probe or primer is described as “corresponding to” a polynucleotide of the present invention, including one of the sequences set out as SEQ ID NOS: 1-29, 57-80, 105, 107, 109-113, 119-128, 139-143, 149-933 and 955, or a variant, if the oligonucleotide probe or primer, or its complement, is contained within one of the sequences set out as SEQ ID NOS: 1-29, 57-80, 105, 107, 109-113, 119-128, 139-143, 149-933 and 955, or a variant of one of the specified sequences.

Two single stranded sequences are said to be substantially complementary when the nucleotides of one strand, optimally aligned and compared, with the appropriate nucleotide insertions and/or deletions, pair with at least 80%, preferably at least 90% to 95%, and more preferably at least 98% to 100%, of the nucleotides of the other strand. Alternatively, substantial complementarity exists when a first DNA strand will selectively hybridize to a second DNA strand under stringent hybridization conditions. Stringent hybridization conditions for determining complementarity include salt conditions of less than about 1 M, more usually less than about 500 mM, and preferably less than about 200 mM. Hybridization temperatures can be as low as 5° C., but are generally greater than about 22° C., more preferably greater than about 30° C., and most preferably greater than about 37° C. Longer DNA fragments may require higher hybridization temperatures for specific hybridization. Since the stringency of hybridization may be affected by other factors such as probe composition, presence of organic solvents and extent of base mismatching, the combination of parameters is more important than the absolute measure of any one alone. The DNA from plants or samples or products containing plant material can be either genomic DNA or DNA derived by preparing cDNA from the RNA present in the sample.

In addition to DNA-DNA hybridization, DNA-RNA or RNA-RNA hybridization assays are also possible. In the first case, the mRNA from expressed genes would then be detected instead of genomic DNA or cDNA derived from mRNA of the sample. In the second case, RNA probes could be used. In addition, artificial analogs of DNA hybridizing specifically to target sequences could also be used.

In specific embodiments, the oligonucleotide probes and/or primers comprise at least about 6 contiguous residues, more preferably at least about 10 contiguous residues, and most preferably at least about 20 contiguous residues complementary to a polynucleotide sequence of the present invention. Probes and primers of the present invention may be from about 8 to 100 base pairs in length, preferably from about 10 to 50 base pairs in length, and more preferably from about 15 to 40 base pairs in length. The probes can be easily selected using procedures well known in the art, taking into account DNA-DNA hybridization stringencies, annealing and melting temperatures, potential for formation of loops, and other factors that are well known in the art. Preferred techniques for designing PCR primers are disclosed in Dieffenbach and Dyksler, PCR Primer: a laboratory manual, CSHL Press: Cold Spring Harbor, N.Y., 1 995. A software program suitable for designing probes, and e specially for designing PCR primers, is available from Premier Biosoft International, 3786 Corina Way, Palo Alto, Calif. 94303-4504.

A plurality of oligonucleotide probes or primers corresponding to a polynucleotide of the present invention may be provided in a kit form. Such kits generally comprise multiple DNA or oligonucleotide probes, each probe being specific for a polynucleotide sequence. Kits of the present invention may comprise one or more probes or primers corresponding to a polynucleotide of the present invention, including a polynucleotide sequence identified in SEQ ID NOS: 1-29, 57-80, 105, 107, 109-113, 119-128, 139-143, 149-933 and 955.

In one embodiment, the present invention provides genetic constructs that include an open reading frame coding for at least a functional portion of a polypeptide of the present invention or a variant thereof. As used herein, the “functional portion” of a polypeptide is that portion which contains the active site essential for affecting the metabolic step, i.e., the portion of the molecule that is capable of binding one or more reactants or is capable of improving or regulating the rate of reaction. The functional portion can be determined by targeted mutagenesis and screening of modified protein products with protocols well known in the art. Normally, the functional portion is 10-20 amino acids, but can be shorter or longer. The active site may be made up of separate portions present on one or more polypeptide chains and will generally exhibit high substrate specificity.

The open reading frame may be inserted in the genetic construct in a sense or antisense orientation, such that transformation of a target plant with the genetic construct will produce a change in the amount or structure of the polypeptide compared to the wild-type plant. Transformation with a genetic construct comprising an open reading frame in a sense orientation will generally result in modified expression of the selected gene, while transformation with a genetic construct comprising an open reading frame in an antisense orientation also generally results in modified expression of the selected gene. A population of plants transformed with a genetic construct comprising an open reading frame of the present invention in either a sense or antisense orientation may be screened for increased or reduced expression of the gene in question using techniques well known to those of skill in the art, and plants having the desired phenotypes may thus be isolated.

Alternatively, expression of a gene involved in the biosynthesis of polysaccharides may be inhibited by inserting a portion of an open reading frame of the present invention, in either sense or antisense orientation, in the genetic construct. Such portions need not be full-length but preferably comprise at least 25 and more preferably at least 50 residues of a polynucleotide of the present invention. A much longer portion or even the full length polynucleotide corresponding to the complete open reading frame may be employed. The portion of the open reading frame does not need to be precisely the same as the endogenous sequence, provided that there is sufficient sequence similarity to achieve inhibition of the target gene. Thus a sequence derived from one species may be used to inhibit expression of a gene in a different species.

In a second embodiment, the inventive genetic constructs comprise a polynucleotide including a non-coding region of a polynucleotide of the present invention, or a polynucleotide sequence complementary to such a non-coding region. As used herein the term “non-coding region” includes both transcribed sequences that are not translated, and non-transcribed sequences within about 2000 base pairs 5′ or 3′ of the translated sequences or open reading frames. Examples of non-coding regions that may be usefully employed in the inventive constructs include introns and 5′-non-coding leader sequences. Transformation of a target plant with such a genetic construct may lead to a reduction in the amount of polysaccharide synthesized by the plant by the process of co-suppression, in a manner similar to that discussed, for example, by Napoli et al. (Plant Cell 2:279-290, 1990) and de Carvalho Niebel et al. (Plant Cell 7:347-358, 1995).

Alternatively, regulation of polysaccharide synthesis can be achieved by inserting appropriate sequences or subsequences (e.g., DNA or RNA) in ribozyme constructs (McIntyre, Manners, Transgenic Res. 5(4):257-262, 1996). Ribozymes are synthetic RNA molecules that comprise a hybridizing region complementary to two regions, each of which comprises at least 5 contiguous nucleotides in a mRNA molecule encoded by one of the inventive polynucleotides. Ribozymes possess highly specific endonuclease activity, which autocatalytically cleaves the mRNA.

The genetic constructs of the present invention further comprise a gene promoter sequence and a gene termination sequence, operably linked to the DNA sequence to be transcribed, which control expression of the gene. The gene promoter sequence is generally positioned at the 5′ end of the DNA sequence to be transcribed, and is employed to initiate transcription of the DNA sequence. Gene promoter sequences are generally found in the 5′ non-coding region of a gene but they may exist downstream of the open reading frame, in introns (Luehrsen, Mol. Gen. Genet. 225:81-93, 1991) or in the coding region, as for example in a plant defence gene (Douglas et al., EMBO J. 10:1767-1775, 1991). When the construct includes an open reading frame in a sense orientation, the gene promoter sequence also initiates translation of the open reading frame. For DNA constructs comprising either an open reading frame in an antisense orientation or a non-coding region, the gene promoter sequence consists only of a transcription initiation site having a RNA polymerase binding site.

A variety of gene promoter sequences which may be usefully employed in the DNA constructs of the present invention are well known in the art. The gene promoter sequence, and also the gene termination sequence, may be endogenous to the target plant host or may be exogenous, provided the promoter is functional in the target host. For example, the promoter and termination sequences may be from other plant species, plant viruses, bacterial plasmids, and the like. Preferably, gene promoter and termination sequences are from the inventive sequences themselves.

Factors influencing the choice of promoter include the desired tissue specificity of the construct, and the timing of transcription and translation. For example, constitutive promoters, such as the 35S Cauliflower Mosaic Virus (CaMV 35S) promoter, will affect the activity of the enzyme in all parts of the plant. Use of a tissue specific promoter will result in production of the desired sense or antisense RNA only in the tissue of interest. With genetic constructs employing inducible gene promoter sequences, the rate of RNA polymerase binding and initiation can be modulated by external stimuli, such as light, heat, anaerobic stress, alteration in nutrient conditions and the like. Temporally regulated promoters can be employed to effect modulation of the rate of RNA polymerase binding and initiation at a specific time during development of a transformed cell. Preferably, the original promoters from the enzyme gene in question, or promoters from a specific tissue-targeted gene in the organism to be transformed, such as eucalyptus or pine are used. Other examples of gene promoters which may be usefully employed in the present invention include mannopine synthase (mas), octopine synthase (ocs), and those reviewed by Chua et al. (Science 244:174-181, 1989).

The gene termination sequence, which is located 3′ to the DNA sequence to be transcribed, may come from the same gene as the gene promoter sequence or may be from a different gene. Many gene termination sequences known in the art may be usefully employed in the present invention, such as the 3′ end of the Agrobacterium tumefaciens nopaline synthase gene. However, preferred gene terminator sequences are those from the original enzyme gene or from the target species to be transformed.

The genetic constructs of the present invention may also contain a selection marker that is effective in plant cells, to allow for the detection of transformed cells containing the inventive construct. Such markers, which are well known in the art, typically confer resistance to one or more toxins. One example of such a marker is the NPTII gene whose expression results in resistance to kanamycin or hygromycin, antibiotics which are usually toxic to plant cells at a moderate concentration (Rogers et al., in Weissbach, A and H, eds., Methods for Plant Molecular Biology, Academic Press Inc.: San Diego, Calif., 1988). Transformed cells can thus be identified by their ability to grow in media containing the antibiotic in question. Alternatively, the presence of the desired construct in transformed cells can be determined by means of other techniques well known in the art, such as Southern and Western blots.

A transcription initiation site is additionally included in the genetic construct when the sequence to be transcribed lacks such a site.

Techniques for operatively linking the components of the inventive genetic constructs are well known in the art and include the use of synthetic linkers containing one or more restriction endonuclease sites as described, for example, by Sambrook and Russell (Molecular cloning: a laboratory manual, Third Edition, CSHL Press: Cold Spring Harbor, N.Y., 2001). The genetic construct of the present invention may be linked to a vector having at least one replication system, for example E. coli, whereby after each manipulation, the resulting construct can be cloned and sequenced and the correctness of the manipulation determined.

The genetic constructs of the present invention may be used to transform a variety of plants, both monocotyledonous (e.g., grasses, corn, grains, oat, wheat and barley), dicotyledonous (e.g., Arabidopsis, tobacco, legumes, alfalfa, oaks, eucalyptus, maple), and Gymnosperms (e.g., Scots pine; see Aronen, Finnish Forest Res. Papers, Vol. 595, 1996), white spruce (Ellis et al., Biotechnology 11:84-89, 1993), and larch (Huang et al., In Vitro Cell 27:201-207, 1991). In a preferred embodiment, the inventive genetic constructs are employed to transform woody plants, herein defined as a tree or shrub whose stem lives for a number of years and increases in diameter each year by the addition of woody tissue. Preferably the target plant is selected from the group consisting of eucalyptus and pine species, most preferably from the group consisting of Eucalyptus grandis and Pinus radiata. Other species which may be usefully transformed with the genetic constructs of the present invention include, but are not limited to: pines such as Pinus banksiana, Pinus brutia, Pinus caribaea, Pinus clausa, Pinus contorta, Pinus coulteri, Pinus echinata, Pinus eldarica, Pinus ellioti, Pinus jeffreyi, Pinus lambertiana, Pinus monticola, Pinus nigra, Pinus palustrus, Pinus pinaster, Pinus ponderosa, Pinus resinosa, Pinus rigida, Pinus serotina, Pinus strobus, Pinus sylvestris, Pinus taeda, Pinus virginiana; other gymnosperms, such as Abies amabilis, Abies balsamea, Abies concolor, Abies grandis, Abies lasiocarpa, Abies magnifica, Abies procera, Chamaecyparis lawsoniona, Chamaecyparis nootkatensis, Chamaecyparis thyoides, Juniperus virginiana, Larix decidua, Larix laricina, Larix leptolepis, Larix occidentalis, Larix siberica, Libocedrus decurrens, Picea abies, Picea engelmanni, Picea glauca, Picea mariana, Picea pungens, Picea rubens, Picea sitchensis, Pseudotsuga menziesii, Sequoia gigantea, Sequoia sempervirens, Taxodium distichum, Tsuga canadensis, Tsuga heterophylla, Tsuga mertensiana, Thuja occidentalis, Thuja plicata; and Eucalypts, such as Eucalyptus alba, Eucalyptus bancroftii, Eucalyptus botyroides, Eucalyptus bridgesiana, Eucalyptus calophylla, Eucalyptus camaldulensis, Eucalyptus citriodora, Eucalyptus cladocalyx, Eucalyptus coccifera, Eucalyptus curtisii, Eucalyptus dalrympleana, Eucalyptus deglupta, Eucalyptus delagatensis, Eucalyptus diversicolor, Eucalyptus dunnii, Eucalyptus ficifolia, Eucalyptus globulus, Eucalyptus gomphocephala, Eucalyptus gunnii, Eucalyptus henryi, Eucalyptus laevopinea, Eucalyptus macarthurii, Eucalyptus macrorhyncha, Eucalyptus maculata, Eucalyptus marginata, Eucalyptus megacarpa, Eucalyptus melliodora, Eucalyptus nicholii, Eucalyptus nitens, Eucalyptus nova-anglica, Eucalyptus obliqua, Eucalyptus obtusiflora, Eucalyptus oreades, Eucalyptus pauciflora, Eucalyptus polybractea, Eucalyptus regnans, Eucalyptus resinifera, Eucalyptus robusta, Eucalyptus rudis, Eucalyptus saligna, Eucalyptus sideroxylon, Eucalyptus stuartiana, Eucalyptus tereticornis, Eucalyptus torelliana, Eucalyptus urnigera, Eucalyptus urophylla, Eucalyptus viminalis, Eucalyptus viridis, Eucalyptus wandoo and Eucalyptus youmanni; together with hybrids of the above species.

As discussed above, transformation of a plant with a genetic construct of the present invention will result in a modification in polysaccharide synthesis in the plant. For example, an increase in the production of cellulose in a plant may be obtained by introducing a genetic construct comprising an open reading frame encoding the enzyme CEL in a sense orientation. Similarly, transformation of a plant with a genetic construct comprising either an open reading frame encoding CEL in an antisense orientation or a non-coding (untranslated) region of a CEL gene will lead to a reduction in the cellulose content of the transformed plant.

Techniques for stably incorporating genetic constructs into the genome of target plants are well known in the art and include Agrobacterium tumefaciens mediated introduction, electroporation, protoplast fusion, injection into reproductive organs, injection into immature embryos, high velocity projectile introduction and the like. The choice of technique will depend upon the target plant to be transformed. For example, dicotyledonous plants and certain monocots and gymnosperms may be transformed by Agrobacterium Ti plasmid technology, as described, for example by Bevan (Nucleic Acids Res. 12:8711-8721, 1984). Targets for the introduction of the genetic constructs of the present invention include tissues, such as leaf tissue, disseminated cells, protoplasts, seeds, embryos, meristematic regions; cotyledons, hypocotyls, and the like. The preferred method for transforming eucalyptus and pine is a biolistic method using pollen (see, for example, Aronen, Finnish Forest Res. Papers, 595:53, 1996) or easily regenerable embryonic tissues.

Once the cells are transformed, cells having the inventive genetic construct incorporated in their genome may be selected by means of a marker, such as the kanamycin resistance marker discussed above. Transgenic cells may then be cultured in an appropriate medium to regenerate whole plants, using techniques well known in the art. In the case of protoplasts, the cell wall is allowed to reform under appropriate osmotic conditions. In the case of seeds or embryos, an appropriate germination or callus initiation medium is employed. For explants, an appropriate regeneration medium is used. Regeneration of plants is well established for many species. For a review of regeneration of forest trees, see Dunstan et al., “Somatic embryogenesis in woody plants,” in Thorpe TA, ed., In vitro embryogenesis of plants, (Current Plant Science and Biotechnology in Agriculture), Vol. 20, Chapter 12, pp. 471-540, 1995. Specific protocols for the regeneration of spruce are discussed by Roberts et al., (“Somatic embryogenesis of spruce,” in Redenbaugh K, ed., Synseed: applications of synthetic seed to crop improvement, Chapter 23, pp. 427-449, CRC Press: [n.p.], 1993). The resulting transformed plants may be reproduced sexually or asexually, using methods well known in the art, to give successive generations of transgenic plants.

As discussed above, the production of RNA in target plant cells can be controlled by choice of the promoter sequence, or by selecting the number of functional copies or the site of integration of the polynucleotides incorporated into the genome of the target plant host. A target plant may be transformed with more than one genetic construct of the present invention, thereby modulating the activity of more than one cell wall polysaccharide enzyme, affecting enzyme activity in more than one tissue, or affecting enzyme activity at more than one expression time. Similarly, a genetic construct may be assembled containing more than one open reading frame coding for a polypeptide encoded by a polynucleotide of the present invention or more than one non-coding region of a gene coding for such a polypeptide. The polynucleotides of the present inventive may also be employed in combination with other known sequences encoding polypeptides involved in the synthesis of cell wall polysaccharides. In this manner, it may be possible to modify a biosynthetic pathway of cell wall polysaccharides in a non-woody plant to produce a new type of woody plant.

Polynucleotides of the present invention may also be used to spacifically suppress gene expression by methods that operate post-transcriptionally to block the synthesis of products of targeted genes, such as RNA interference (RNAi), and quelling. For a review of techniques of gene suppression see Science, 288:1370-1372, 2000. Exemplary gene silencing methods are also provided in WO 99/49029 and WO 99/53050. Posttranscriptional gene silencing is brought about by a sequence-specific RNA degradation process that results in the rapid degradation of transcripts of sequence-related genes. Studies have provided evidence that double-stranded RNA may act as a mediator of sequence-specific gene silencing (see, e.g., review by Montgomery and Fire, Trends in Genetics, 14: 255-258, 1998). Gene constructs that produce transcripts with self-complementary regions are particularly efficient at gene silencing. A unique feature of this posttranscriptional gene silencing pathway is that silencing is not limited to the cells where it is initiated. The gene-silencing effects may be disseminated to other parts of an organism and even transmitted through the germ line to several generations.

The polynucleotides of the present invention may be employed to generate gene silencing constructs and or gene-specific self-complementary RNA sequences that can be delivered by conventional art-known methods to plant tissues, such as Eucalyptus and Pinus tissues. Within genetic constructs, sense and antisense sequences can be placed in regions flanking an intron sequence in proper splicing orientation with donor and acceptor splicing sites, such that intron sequences are removed during processing of the transcript and sense and antisense sequences, as well as splice junction sequences, bind together to form double-stranded RNA. Alternatively, spacer sequences of various lengths may be employed to separate self-complementary regions of sequence in the construct. During processing of the gene construct transcript, intron sequences are spliced-out, allowing sense and anti-sense sequences, as well as splice junction sequences, to bind forming double-stranded RNA. Select ribonucleases bind to and cleave the double-stranded RNA, thereby initiating the cascade of events leading to degradation of specific mRNA gene sequences, and silencing specific genes. Alternatively, rather than using a gene construct to express the self-complementary RNA sequences, the gene-specific double-stranded RNA segments are delivered to one or more targeted areas to be internalized into the cell cytoplasm to exert a gene silencing effect. Gene silencing RNA sequences comprising the polynucleotides of the present invention are useful for creating genetically modified plants with desired phenotypes as well as for characterizing genes (e.g., in high-throughput screening of sequences), and studying their functions in intact organisms.

The following examples are offered by way of illustration and not by way of limitation.

### EXAMPLE 1

Isolation and Characterization of cDNA Clones from Eucalyptus grandis and Pinus radiata

Eucalyptus grandis cDNA expression libraries (from various tissues, including flowers, leaves, phloem, roots, seeds, shoot buds and xylem), and Pinus radiata cDNA expression libraries (from various tissues, including cell cultures, fascicle meristems, phloem, pollen sacs, roots, seedlings, shoot buds, strobilus and xylem) were constructed and screened as follows.

mRNA was extracted from the plant tissue using the protocol of Chang et al. (Plant Molecular Biology Reporter 11:113-116, 1993) with minor modifications. Specifically, samples were dissolved in CPC-RNAXB (100 mM Tris-Cl, pH 8.0; 25 mM EDTA; 2.0 M NaCl; 2%CTAB; 2% PVP and 0.05% Spermidine*3HCl) and extracted with chloroform: isoamyl alcohol, 24:1. mRNA was precipitated with ethanol and the total RNA preparate was purified using a Poly(A) Quik mRNA Isolation Kit (Stratagene, La Jolla, Calif.). A cDNA expression library was constructed from the purified mRNA by reverse transcriptase synthesis followed by insertion of the resulting cDNA clones in Lambda ZAP using a ZAP Express cDNA Synthesis Kit (Stratagene), according to the manufacturer's protocol. The resulting cDNAs were packaged using a Gigapack II Packaging Extract (Stratagene) employing 1 μl of sample DNA from the 5 μl ligation mix. Mass excision of the library was done using XL1-Blue MRF′ cells and XLOLR cells (Stratagene) with ExAssist helper phage (Stratagene). The excised phagemids were diluted with NZY broth (Gibco BRL, Gaithersburg, Md.) and plated out onto LB-kanamycin agar plates containing X-gal and isopropylthio-beta-galactoside (IPTG).

Of the colonies plated and picked for DNA miniprep, 99% contained an insert suitable for sequencing. Positive colonies were cultured in NZY broth with kanamycin and cDNA was purified by means of alkaline lysis and polyethylene glycol (PEG) precipitation. Agarose gel at 1% was used to screen sequencing templates for chromosomal contamination. Dye primer sequences were prepared using a Turbo Catalyst 800 machine (Perkin Elmer/Applied Biosystems Division, Foster City, Calif.) according to the manufacturer's protocol.

DNA sequences for positive clones were obtained using a Perkin Elmer/Applied Biosystems Division Prism 377 sequencer. cDNA clones were sequenced first from the 5′ end and, in some cases, also from the 3′ end. For some clones, internal sequence was obtained using subcloned fragments. Subcloning was performed using standard procedures of restriction mapping and subcloning to pBluescript II SK+ vector.

The determined cDNA sequences are provided in SEQ ID NO: 1-29, 57-80, 105, 107, 109-113, 119-128, 139-143, 149-933 and 955. SEQ ID NO: 1, 4, 5, 8, 10, 11, 16, 17, 20, 22, 24, 25, 27, 59, 67-70, 74-77, 80, 109-113, 140, 143, 162-185, 196, 199-204, 234-251, 257-263, 265-272, 294-318, 331-365, 367-372, 378-381, 386-389, 394-398, 435-481, 504-506, 513-521, 529, 530, 548-551, 555, 574-586, 590, 591, 613-620, 772-911, 919, 920, 923, 925, 926, 928, 931, and 932 were isolated from Pinus radiata, with SEQ ID NO: 2, 3, 6, 7, 9, 12-15, 18, 19, 21, 23, 26, 28, 29, 57, 58, 60-66, 71-73, 78, 79, 105, 107, 119-128, 139, 141, 142, 149-161, 186-195, 197, 198, 205-233, 252-256, 264, 273-293, 319-330, 366, 373-377, 382-385, 390-393, 399-434, 479-503, 507-512, 522-528, 531-547, 552-554, 556-573, 587-589, 592-612, 621-771, 912, 913-918, 921, 922, 924, 927, 929, 930, 933 and 955 being isolated from Eucalyptus grandis.

### Example 2

### Polynucleotide and Amino Acid Analysis

The determined cDNA sequences described above were compared to and aligned with known sequences in the EMBL database (as updated to May 1999). Specifically, the polynucleotides identified in SEQ ID NO: 1-29, 57-80, 105, 107, 109-113, 119-128, 139-143 and 149-933 were compared to polynucleotides in the EMBL database using the BLASTN algorithm Version 2.0.6 [Sep-16-1998] set to the following running parameters: Unix running command: blastall -p blastn -d embldb -e 10-G0 -E0 -r1-v30 -b30 -i queryseq -o results. Multiple alignments of redundant sequences were used to build up reliable consensus sequences. Based on similarity to known sequences from other plant or non-plant species, the isolated polynucleotides of the present invention identified as SEQ ID NO: 1-29, 57-80, 105, 107, 109-113, 119-128, 139-143, 149-933 and 955 were identified as encoding the enzymes shown in Table 1, above.

SEQ ID NO: 152 is a corrected version (without linker) of SEQ ID NO: 141 and an extension of SEQ ID NO: 107. The amino acid sequence encoded by the open reading frame of SEQ ID NO: 152 is provided in SEQ ID NO: 146. SEQ ID NO: 955 is a corrected version of SEQ ID NO: 141, which was determined to contain a frameshift. The amino acid sequence encoded by SEQ ID NO: 955 is provided in SEQ ID NO: 956.

The cDNA sequences of SEQ ID NO: 58, 60, 62, 64, 65, 67-70, 72, 74, 75, 77, 78, 80, 105, 107, 119-121, 123-128 and 139-143 were determined to have less than 40% identity to sequences in the EMBL database using the computer algorithm BLASTN, as described above. The cDNA sequences of SEQ ID NO: 57, 59, 66, 79 and 122 were determined to have less than 60% identity to sequences in the EMBL database using BLASTN, as described above. The cDNA sequences of SEQ ID NO: 61, 71, 73 and 76 were determined to have less than 75% identity to sequences in the EMBL database using BLASTN, as described above. The cDNA sequence of SEQ ID NO: 63 was determined to have less than 90% identity to sequences in the EMBL database using BLASTN, as described above.

### Example 3

### Functional Identification of Cellulose Biosynthetic Genes

Sense constructs containing sequences including the coding regions for UGP (SEQ ID NO: 79) and SUS (SEQ ID NO: 105) from Eucalyptus grandis, and UGP (SEQ ID NO: 80) from Pinus radiata were inserted into the expression vector pET16b (Clontech Laboratories Inc, Palo Alto, Calif.). The resulting constructs were transformed into E. coli XL1-Blue (Stratagene) and induced to produce recombinant protein by the addition of IPTG. Purified proteins were obtained using Ni²⁺ column chromatography (Janknecht et al., Proc. Natl. Acad. Sci. USA, 88:8972-8976, 1991). Enzyme assays for each of the purified proteins demonstrated the expected substrate specificity and enzymatic activity for the genes tested.

Enzyme assays for UGP were performed using published methods (Peng and Chang, FEBS Lett. 329:153-158, 1993). The data shown in Table 2 demonstrates enzyme activity for the expressed proteins as compared to data from Katsube et al., Biochem. 30:8546-8551, 1991 and Nakano et al., J. Biochem. 106:528-532, 1989.

TABLE 2

| | | | | |
|---|---|---|---|---|
| SEQ ID NO: | 80 | 79 | Katsube et al. | Nakano et al. |
| Species | P. radiata | E. grandis | S. tuberosum | S. tuberosum |
| Enzyme | UGP | UGP | UGP | UGP |
| K_{M}^{G1P} | 0.121 | 0.126 | 0.130 | 0.180 |
| SEM | 0.020 | 0.002 | n.a. | n.a. |
| K_{M}^{UTP} | 0.091 | not done | 0.076 | 0.170 |
| SEM | 0.015 | not done | n.a. | n.a. |
| K_{M}^{ATP} | no activity | no activity | no activity | no activity |

Enzyme assays for SUS (sucrose synthase) were performed using the methods described by Sebkova et al. Plant Physiol., 108:75-83, 1995. The data shown in Table 3 demonstrates enzyme activity for the expressed proteins. The K_{M}^{Sucrose} of E. grandis is compared with the data reported by Delmer, J. Biol. Chem. 247:3822-3828, 1972 and Nakai et al., Biosci. Biotech. Biochem. 61:1500-1503, 1997.

TABLE 3

| | | | |
|---|---|---|---|
| SEQ ID NO: | 105 | Delmer et al. | Nakai et al. |
| Species | E. grandis | V. radiata | V. radiata |
| Enzyme | SUS | SUS | SUS |
| K_{M}^{Sucrose} | 1.651 | 16.700 | 161.000 |
| SEM | 0.371 | n.a. | n.a. |
| K_{M}^{UDP} | 0.028 | n.a. | n.a. |
| SEM | 0.003 | n.a. | n.a. |

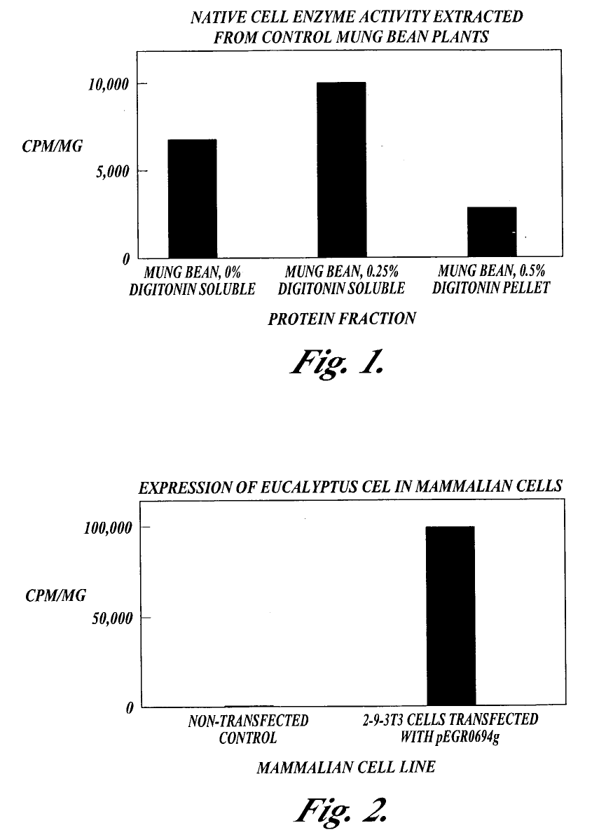

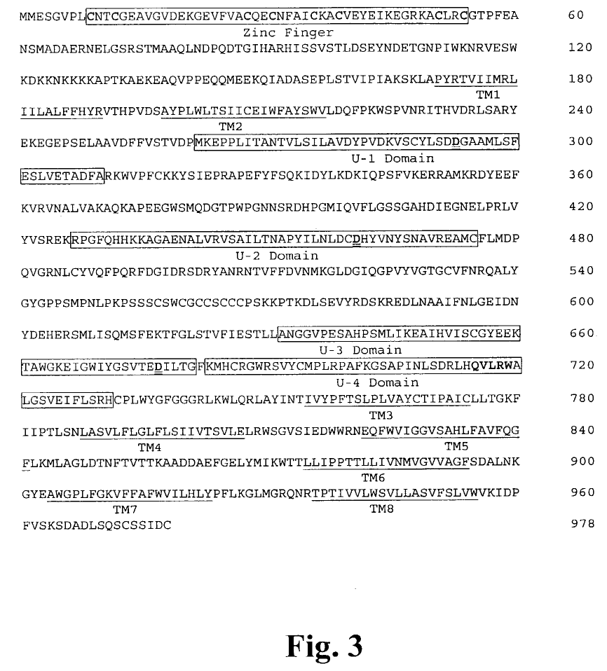

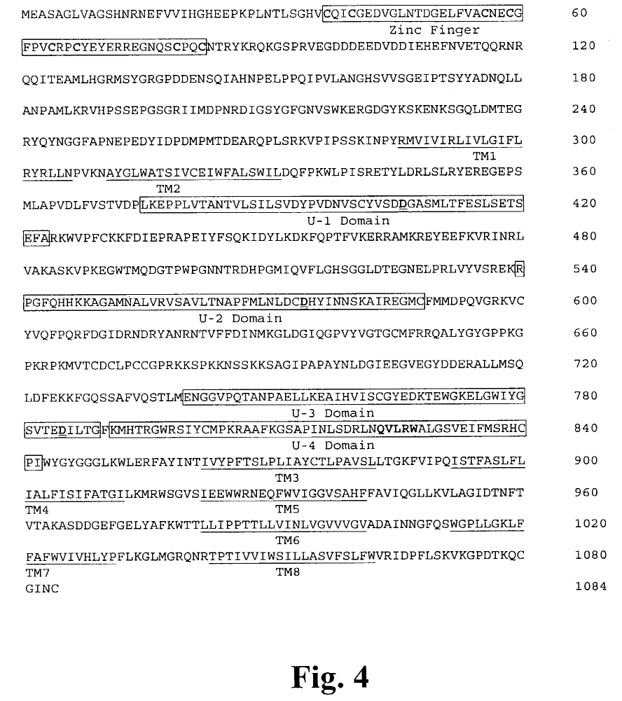

A sense construct containing the sequence of the coding region for cellulose synthase (CEL; SEQ ID NO: 107) from Eucalyptus grandis was inserted into the protein expression vector pcDNA3 (Invitrogen, Carlsbad, Calif.). The resulting construct was transfected into mammalian 293T cells (DuBridge RB et al., Mol. Cell. Biol. 7:379-387, 1987), and recombinant protein was induced by the addition of IPTG. Proteins were solubilized from membranes, and the level of CEL activity was determined as described by Kudlicka and Brown, Plant Phys. 115:643-656, 1997. As a positive control for activity, native CEL enzyme was solubilized from mung bean (Vigna radiata) plants. The determined levels of CEL activity for V. radiata are shown in FIG. 1. The levels of CEL activity found in mammalian 293T cells transfected with the Eucalyptus CEL expression clone were found to be similar to those obtained from V. radiata (FIG. 2). CEL activity was absent in non-transfected control 293T cells.

### EXAMPLE 4

### Use of a Cellulose Synthase (CEL) Gene to Modify Polysaccharide Biosynthesis

Transformation of tobacco plants with a Pinus radiata CEL gene is performed as follows. Genetic constructs comprising sense and anti-sense constructs containing a polynucleotide including the coding region of CEL (SEQ ID NO: 8) from Pinus radiata are constructed and inserted into Agrobacterium tumefaciens by direct transformation using published methods (See, An G, Ebert PR, Mitra A, Ha SB, “Binary Vectors,” in Gelvin SB and Schilperoort R A, eds., Plant Molecular Biology Manual, Kluwer Academic Publishers: Dordrecht, 1988). The constructs of sense polynucleotides are made by cloning PBK-CMV plasmid cDNA inserts into pART7 plasmids, followed by cloning of the NotI-digested 35S-Insert-OCS 3′UTR-fragments from the pART7 vectors into pART27 plant expression vectors (See Gleave A, “A versatile binary vector system with a T-DNA organizational structure conducive to efficient integration of cloned DNA into the plant genome,” Plant Molecular Biology 20:1203-1207, 1992). The presence and integrity of the transgenic constructs are verified by restriction digestion and DNA sequencing.

Tobacco (Nicotiana tabacum cv. Samsun) leaf sections are transformed with the sense and anti-sense CEL constructs using the method of Horsch et al., Science 227:1229-1231, 1985. Transformed plants containing the appropriate CEL construct are verified using Southern blot experiments. Expression of Pinus CEL in transformed plants is confirmed by isolating total RNA from each independent transformed plant line created with the CEL sense and anti-sense constructs. The RNA samples are analysed in Northern blot experiments to determine the level of expression of the transgene in each transformed line.

The total activity of CEL enzyme, encoded by the Pinus CEL gene and by the endogenous tobacco CEL gene, is analysed for each transformed plant line created with the CEL sense and anti-sense constructs. Crude protein extracts are prepared from each transformed plant and assayed using the methods of Robertson et al., Biochem J. 306:745-750, 1995 and Pear et al., Proc. Natl. Acad. Sci. USA 93:12637-12642, 1996.

The concentration of cellulose in the transformed tobacco plants is determined using the method of Smith and Harris, Plant Phys. 107:1399-1409, 1995. Briefly, whole tobacco plants, of an average age of 38 days, are frozen in liquid nitrogen and ground to a fine powder in a mortar and pestle. The cellulose content of 100 mg of frozen powder from an empty vector-transformed control plant line, at least one independent transformed plant line containing the sense construct for CEL and at least one independent transformed plant lines containing the anti-sense construct for CEL are determined using a glucan estimation kit from Megazyme (Warriewood, New South Wales, Australia) using the protocols supplied by the manufacturer.

SEQ ID NOS: 1-956 are set out in the attached Sequence Listing. The codes for nucleotide and amino acid sequences used in the attached Sequence Listing, including the symbols “n” and “Xaa”, conform to WIPO Standard ST.25 (1998), Appendix 2, Table 1.

Although the present invention has been described in some detail by way of illustration and example for purposes of clarity of understanding, changes and modifications can be carried out without departing from the scope of the invention which is intended to be limited only by the scope of the claims.

We claim:

1. An isolated polynucleotide comprising a nucleotide sequence selected from the group consisting of: SEQ ID NOS: 1-29, 57-80, 105, 107, 109-113, 119-128, 139-143, 149-933 and 955.

2. An isolated polynucleotide comprising a nucleotide sequence selected from the group consisting of:
(a) complements of the sequences recited in SEQ ID NOS: 1-29, 57-80, 105, 107, 109-113, 119-128, 139-143, 149-933 and 955;
(b) reverse complements of the sequences recited in SEQ ID NOS: 1-29, 57-80, 105, 107, 109-113, 119-128, 139-143, 149-933 and 955; and
(c) reverse sequences of the sequences recited in SEQ ID NOS: 1-29, 57-80, 105, 107, 109-113, 119-128, 139-143, 149-933 and 955;

3. An isolated polynucleotide comprising a nucleotide sequence selected from the group consisting of:
(a) nucleotide sequences having at least 75% identity to a nucleotide sequence recited in SEQ ID NOS: 1-29, 57-80, 105, 107, 109-113, 119-128, 139-143, 149-933 and 955;
(b) nucleotide sequences having at least 90% identity to a nucleotide sequence recited in SEQ ID NOS: 1-29, 57-80, 105, 107, 109-113, 119-128, 139-143, 149-933 and 955; and
(c) nucleotide sequences having at least 95% identity to a nucleotide sequence recited in SEQ ID NOS: 1-29, 57-80, 105, 107, 109-113, 119-128, 139-143, 149-933 and 955,
wherein the polynucleotide encodes a polypeptide having substantially the same functional properties as a polypeptide encoded by SEQ ID NO: 1-29, 57-80, 105, 107, 109-113, 119-128, 139-143, 149-933 and 955.

4. An isolated polynucleotide comprising a nucleotide sequence selected from the group consisting of:
(a) nucleotide sequences that hybridize to a sequence recited in SEQ ID NO: 1-29, 57-80, 105, 107, 109-113, 119-128, 139-143, 149-933 and 955 under stringent hybridization conditions;
(b) nucleotide sequences that are 200-mers of a sequence recited in SEQ ID NO: 1-29, 57-80, 105, 107, 109-113, 119-128, 139-143, 149-933 and 955;
(c) nucleotide sequences that are 100-mers of a sequence recited in SEQ ID NO: 1-29, 57-80, 105, 107, 109-113, 119-128, 139-143, 149-933 and 955;
(d) nucleotide sequences that are 40-mers of a sequence recited in SEQ ID NO: 1-29, 57-80, 105, 107, 109-113, 119-128, 139-143, 149-933 and 955;
(e) nucleotide sequences that are 20-mers of a sequence recited in SEQ ID NO: 1-29, 57-80, 105, 107, 109-113, 119-128, 139-143, 149-933 and 955; and
(f) nucleotide sequences that are degeneratively equivalent to a sequence recited in SEQ ID NO: 1-29, 57-80, 105, 107, 109-113, 119-128, 139-143, 149-933 and 955.

5. An oligonucleotide probe or primer comprising at least 10 contiguous residues complementary to 10 contiguous residues of a nucleotide sequence recited in SEQ ID NO: 1-29, 57-80, 105, 107, 109-113, 119-128, 139-143, 149-933 and 955.

6. A kit comprising a plurality of oligonucleotide probes or primers of claim 5.

7. A genetic construct comprising a polynucleotide of any one of claims 1-4.

8. A transgenic cell comprising a genetic construct according to claim 7.

9. A genetic construct comprising, in the 5′-3′ direction:
(a) a gene promoter sequence;
(b) a polynucleotide sequence comprising at least one of the following: (1) a polynucleotide of claim 1; (2) a polynucleotide coding for at least a functional portion of a polypeptide encoded by a polynucleotide of claim 1; (3) an open reading frame of a polynucleotide of claim 1; and (4) a polynucleotide sequence comprising a non-coding region of a polynucleotide of claim 1; and
(c) a gene termination sequence.

10. The genetic construct of claim 9, wherein the polynucleotide sequence is in a sense orientation.

11. The genetic construct of claim 9, wherein the polynucleotide sequence is in an antisense orientation.

12. The genetic construct of claim 9, wherein the gene promoter sequence is functional in a plant host to provide for transcription in xylem.

13. A transgenic plant cell comprising a genetic construct of claim 9.

14. A plant comprising a transgenic plant cell according to claim 13, or a part, propagule or progeny thereof.

15. A method for modulating one or more of the polysaccharide content, the polysaccharide composition and the polysaccharide structure of a plant, comprising stably incorporating into the genome of the plant a polynucleotide of any one of claims 1-4.

16. The method of claim 15, wherein the plant is selected from the group consisting of eucalyptus and pine species.

17. A method for producing a plant having one or more of altered polysaccharide content, altered polysaccharide composition and altered polysaccharide structure, comprising:
(a) transforming a plant cell with a genetic construct of claim 9 to provide a transgenic cell; and
(b) cultivating the transgenic cell under conditions conducive to regeneration and mature plant growth.

18. A method for modifying the activity of a polypeptide involved in a polysaccharide biosynthetic pathway in a plant comprising stably incorporating into the genome of the plant a genetic construct of claim 9.

19. An isolated polypeptide comprising an amino acid sequence selected from the group consisting of: SEQ ID NOS: 30-56, 81-104, 106, 108, 114-118, 129-138, 144-148, 934-954 and 956.

20. An isolated polypeptide comprising an amino acid sequence selected from the group consisting of:
(a) sequences having at least 70% identity to a sequence of SEQ ID NOS: 30-56, 81-104, 106, 108, 114-118, 129-138, 144-148,934-954 and 956;
(b) sequences having at least 90% identity to a sequence of SEQ ID NOS: 30-56, 81-104, 106, 108, 114-118, 129-138, 144-148, 934-954 and 956; and
(c) sequences having at least 95% identity to a sequence of SEQ ID NOS: 30-56, 81-104, 106, 108, 114-118, 129-138, 144-148,934-954 and 956,
wherein the polypeptide has substantially the same functional properties as a polypeptide of SEQ ID NOS: 30-56, 81-104, 106, 108, 114-118, 129-138, 144-148, 934-954 and 956.

21. An isolated polypeptide encoded by an isolated polynucleotide sequence of claim 1.
